(19) United States
(12) Patent Application Publication
(10) Pub. No.: US 2020/0032306 A1
(43) Pub. Date: Jan. 30, 2020

(54) Method for the fermentative production of L-lysine

(71) Applicant: Evonik Degussa GmbH, Essen (DE)

(72) Inventors: Frank SCHNEIDER, Halle (DE); Georg Thierbach, Bielefeld (DE); Kornelia Voss, Steinhagen (DE); Thomas Bekel, Halle (Westf.) (DE)

(73) Assignee: Evonik Degussa GmbH, Essen (DE)

(21) Appl. No.: 16/517,045

(22) Filed: Jul. 19, 2019

(30) Foreign Application Priority Data
Jul. 24, 2018 (EP) 18185087.6

(51) Int. Cl. C12P 13/08; C12R 1/15

(52) U.S. Cl. CPC C12P 13/08; C12R 1/15

(57) ABSTRACT

A novel method is used for the fermentative production of L-lysine using bacteria of the species Corynebacterium glutamicum, having the ability to excrete L-lysine, containing in their chromosome a variant of a polynucleotide encoding a polypeptide having hydrolase activity. The bacteria are cultivated in a suitable medium under suitable conditions, and then L-lysine is accumulated in the medium to form an L-lysine containing fermentation broth.

### CROSS-REFERENCE TO RELATED APPLICATIONS

The present application claims the benefit of the European Application EP18185087.6, filed on Jul. 24, 2018, which is incorporated by reference in its entirety.

### REFERENCE TO A SEQUENCE LISTING

The present application is accompanied by an ASCII text file as a computer readable form containing the sequence listing, titled “2019-07-09-seq-list-as-filed,” created on Jul. 9, 2019, 8:42:46 AM, with the file size of 67,749 bytes, which is incorporated by reference in 15 its entirety. Applicants hereby state that the information recorded in computer readable form is identical to the written (on paper or compact disc) sequence listing.

### BACKGROUND OF THE INVENTION

### Field of the Invention

L-lysine is used in human medicine, in the pharmaceutical industry, in the food industry and particularly in nutrition of animals.

### Discussion of the Background

L-lysine is produced by fermentation of strains of the species Corynebacterium glutamicum. Because of the great economic importance, work is continually being done on improving the production methods. Improvements may relate to the fermentation technology such as e.g. stirring and supplying oxygen, or to the composition of the nutrient media e.g. the sugar concentration during fermentation, or to the processing of the fermentation broth to a suitable product form by e.g. drying and granulating the fermentation broth or ion exchange chromatography or may relate to the intrinsic performance properties of the microorganism itself.

The methods used for improving the performance properties of these microorganisms are those of mutagenesis, selection and screening of mutants. The strains obtained in this way are resistant to anti-metabolites or are auxotrophic for metabolites of regulatory importance, and produce L-lysine. A well-known anti-metabolite is the L-lysine analogue S-(2-aminoethyl)-L-cysteine (see e.g. Tosaka et al.: Agricultural and Biological Chemistry 42(4), 745-752, (1978)).

Methods of recombinant DNA technology have likewise been used for a number of years for improvement of L-lysine-producing strains of the species Corynebacterium glutamicum, by modifying, i.e. enhancing or attenuating, individual genes involved in L-lysine biosynthesis and investigating the effect on L-lysine production.

Research on the control of cell lysis of Corynebacterium glutamicum, which may cause decreased productivity during fermentation, is still insufficient. Cell wall hydrolases are known as enzymes that degrade bacterial cell walls, and are present in all microorganisms having peptidoglycan containing cell walls (Rice K C & Bayles K W, Microbiology and Molecular Biology Reviews 2008, 72:85-109). Although research on such cell wall hydrolases has been conducted in various bacteria, their precise control mechanism is still unknown.

The nucleotide sequences of the chromosomes of various bacteria or strains resp. of the species Corynebacterium glutamicum, and their analysis have been disclosed. This information is available at publicly accessible databases and may be used for strain development purposes. One such database is the GenBank data base of the NCBI (National Center for Biotechnology Information, U.S. National Library of Medicine 8600 Rockville Pike, Bethesda Md., 20894 USA).

During the annotation procedure for a sequenced chromosome of an organism identified structures such as e.g. genes or coding sequences are furnished with a unique identifier called locus_tag by the supplier of the information to the data base.

The nucleotide sequence of the Corynebacterium glutamicum ATCC13032 chromosome and its analysis were described by Ikeda and Nakagawa (Applied Microbiology and Biotechnology 62, 99-109 (2003)) and in EP1108790. The information is available at the NCBI under accession number NC_003450. In the chromosome sequence disclosed under accession number NC_003450 locus_tag NCgl1480 identifies a nucleotide sequence starting with a ttg codon coding for a cell wall-associated hydrolase. The amino acid sequence of the encoded polypeptide having a length of 604 amino acids is available under the identifier NP_600753. In EP1108790 A2 the coding sequence is disclosed under sequence 7064 (see GenBank accession number AX127148).

The nucleotide sequence of the Corynebacterium glutamicum ATCC13032 chromosome and its analysis were independently described by Kalinowski et al. (Journal of Biotechnology 104 (1-3), 5-25 (2003)). The information is available at the NCBI under accession number NC_006958. Locus_tag CGTRNA_RS07715 identifies a nucleotide sequence coding for a hydrolase and starting with a ttg codon. The old_locus_tag designation cg1735 is also used in the art. The amino acid sequence of the polypeptide having a length of 604 amino acids is available under the identifier WP_011014435.

The nucleotide sequences of locus_tag NCgl1480 and CGTRNA_RS07715 are identical.

Marchand et al. (Journal of Bacteriology 194(3), 587-597 (2012)) describe NCgl1480 as a member of a group of putative mycomembrane proteins more specifically as a putative cell wall associated hydrolase bearing a signal sequence for the general secretory pathway for cell membrane translocation.

EP3318637 A1 describes the inactivation of various cell wall hydrolases and its effect on L-lysine production. The document further discloses that the inactivation of the cell wall hydrolase encoded by the nucleotide sequence of NCgl11480 by deleting said nucleotide sequence enhanced the production of L-lysine. EP3318637 A1 also suggests a method of replacing the gene encoding the enzyme on the chromosome with a mutated gene so that the enzyme activity can be reduced. However the document does not teach such mutated gene. Accordingly, there is a need to provide such mutated variant(s).

Information concerning transcription signals in Corynebacterium glutamicum, e.g. −10 region of a promoter, or transcriptional start site (TSS) of the gene identified by old_locus_tag cg1735 can be found in Pfeifer-Sancar et al. (BMC Genomics 14:888 (2013)), Albersmeier et al. (Journal of Biotechnology 257 (2017) 99-109) or Mentz et al. (BMC Genomics 2013, 14.714).

The nucleotide sequence of the Corynebacterium glutamicum R chromosome and its analysis was described by Yukawa et al. (Microbiology 153(4): 1042-1058 (2007)). It is available at the NCBI under accession number AP009044. Locus_tag cgR_1596 identifies a nucleotide sequence coding for a hypothetical protein and starting with a gtg codon. The amino acid sequence of the polypeptide having a length of 610 amino acids is available under the identifier BAF54588.

The nucleotide sequence of the Corynebacterium glutamicum R chromosome and its analysis is also available under accession number NC_009342. Under designation cgR_1596 the predicted function of the polypeptide is given as a hydrolase.

The polypeptide identified by locus_tag cgR_1596 of accession number AP009044 is the homologous counterpart of the polypeptide identified by NCgl1480. Amino acid sequence alignment reveals that besides other differences the polypeptide identified by locus_tag cgR_1596 displays 26 additional amino acids at the N-terminus when compared to the polypeptide identified by NCgl1480 (see Table 1).

TABLE 1
Alignment of the amino acid sequence of the N terminus of the polypeptide identified
by locus_tag NCgl1480 according to accession number NC_003450 with the N-terminus of
the polypeptide identified by locus_tag cgR_1596 according to accession number
AP009044.
CLUSTAL multiple sequence alignment

| | |
|---|---|
| cgR_1596 ex AP009044 | MANLDSVRPGVRAALRRTHTQVKGVALTRALIALAVSGALLSSMTPAVAQPQNPDDAAIA |
| NCgl1480 ex NC_003450 | --------------------------MTRALIALAVSGALLSSMTPAVAQPQNPDDAAIA |
| | ********************************** |
| cgR_1596 ex AP009044 | QAEENVSAGDGEVARLAGSLSSTDAEINRVELEMGALREEVNKSLVDLHDAQAIAEQARQ |
| NCgl1480 ex NC_003450 | QAEENVSAGDGEVARLAGSLSSTDAEINRVELEMGALREEVNKSLVDLHDAQAIAEQARQ |
| | ************************************************************ |
| cgR_1596 ex AP009044 | DALAAKKDLDDSQAQIEAAQERLDEISRAAYRQNGTSKGLSGISGNGNSEDALDRQTYLR |
| NCgl1480 ex NC_003450 | DALAAKKDLDDSQAQIEAAQERLDEISRAAYRQNGTSKGLSGISGNGNSEDALDRQTYLR |
| | ************************************************************ |

Tsuge et al. (Journal of Bacteriology 190 (24), 8204-8214, 2008) investigated the effect of deletion of cgR_1596 on cell separation in Corynebacterium glutamicum R. Tsuge et al. further stated that CgR_1596 contains an amino acid sequence domain conserved in cell wall hydrolases and a signal peptide of 49 amino acids.

### SUMMARY OF THE INVENTION

Object of the present invention is to provide new measures for the fermentative production of L-lysine by bacteria of the species Corynebacterium glutamicum.

To achieve the object outlined above the present invention makes available a novel method for the fermentative production of L-lysine using bacteria of the species Corynebacterium glutamicum, having the ability to excrete L-lysine, containing in their chromosome a variant of a polynucleotide encoding the polypeptide shown in SEQ ID NO:2, wherein in the amino acid sequence of the polypeptide, the amino acid at position 90 is isoleucine, the amino acid at position 159 is arginine, the amino acid at position 449 is aspartic acid and the amino acid at position 454 is phenylalanine. The amino acid sequence of said polypeptide variant is also shown under SEQ ID NO:4 of the sequence listing.

The present invention provides a method for the fermentative production of L-lysine comprising the steps of
a) providing a bacterium of the species Corynebacterium glutamicum, having the ability to excrete L-lysine, containing in its chromosome a polynucleotide encoding a polypeptide comprising the amino acid sequence of SEQ ID NO:4,
b) cultivating the bacterium in a suitable medium under suitable conditions, and
c) accumulating the L-lysine in the medium to form an L-lysine containing fermentation broth.

The present invention includes the following embodiments:
1. A method for the fermentative production of L-lysine comprising the steps of
   a) providing a bacterium of the species Corynebacterium glutamicum having the ability to excrete L-lysine containing in its chromosome a polynucleotide encoding a polypeptide comprising the amino acid sequence of SEQ ID NO:4.
   b) cultivating the bacterium in a suitable medium under suitable conditions, and
   c) accumulating said L-lysine in the medium to form an L-lysine containing fermentation broth.
2. The method according to embodiment 1, wherein in the bacterium provided said polynucleotide encoding said amino acid sequence comprises the nucleotide sequence of SEQ ID NO:3 positions 923 to 2812.
3. The method according to embodiment 1, wherein in the bacterium provided the polynucleotide encoding said amino acid sequence comprises the nucleotide sequence of SEQ ID NO:3 positions 923 to 2815.
4. The method according to embodiment 1, wherein in the bacterium provided the polynucleotide encoding said amino acid sequence comprises the nucleotide sequence of SEQ ID NO:3 positions 585 to 2815.
5. The method according to embodiment 1, wherein in the bacterium provided the polynucleotide encoding said amino acid sequence comprises the nucleotide sequence of SEQ ID NO:3 positions 565 to 2815.

### DETAILED DESCRIPTION OF THE INVENTION

It was found that the modified bacteria provided in the method according to the invention excreted L-lysine, into a suitable medium under suitable fermentation conditions in an increased manner with respect to one or more parameters selected from product concentration (e.g. amount of L-lysine produced per volume) as compared to the unmodified bacterium.

It is clear that a higher product concentration facilitates product manufacturing e.g. purification and isolation. An increased product yield reduces the amount of raw material required. An increased product formation rate reduces the time required for a fermentation run thus increasing the availability of a given fermenter.

The method according to the invention thus contributes to the improvement of technical and economic aspects of the manufacturing of L-lysine or L-lysine containing products.

SEQ ID NO:2 of the sequence listing shows the amino acid sequence of a polypeptide encoded by Corynebacterium glutamicum ATCC13032. The difference between the amino acid sequence shown in SEQ ID NO:2 and the amino acid sequence shown under NCgl1480 is a sequence of 26 additional amino acids at the N-terminus of SEQ ID NO:2. The amino acid sequence of SEQ ID NO:2 positions 27 to 630 is identical to the amino acid presented under NCgl1480 with the exception of the interpretation of the ttg start codon in NCgl1480 as a methionine codon. SEQ ID NO:4 of the sequence listing shows the amino acid sequence of a variant of SEQ ID NO:2 used in the method according to the invention. The difference between the two amino acid sequences is the following: The amino acid at position 90 of SEQ ID NO:2 is valine whereas in SEQ ID NO:4 it is isoleucine. The amino acid at position 159 of SEQ ID NO:2 is glycine whereas in SEQ ID NO:4 it is arginine. The amino acid at position 449 of SEQ ID NO:2 is glycine whereas in SEQ ID NO:4 it is aspartic acid. The amino acid at position 454 of SEQ ID NO:2 is serine whereas in SEQ ID NO:4 it is phenylalanine.

Accordingly, using the one letter abbreviation code for amino acids, the variant is characterized by the following amino acid substitutions or amino acid exchanges resp.: V90I, GI59R, G449D and S454F with SEQ ID NO:2 as reference.

Using the information given under NCgl1480, the positions for said amino acid substitutions or amino acid exchanges resp. would be assigned as: V64I, G133R, G423D and S428F.

In a preferred embodiment the bacterium provided in the method according to the invention contains in its chromosome a polynucleotide encoding an amino acid sequence of a polypeptide shown in SEQ ID NO:4 comprising the nucleotide sequence of positions 923 to 2812 of SEQ ID NO:3.

Further in a preferred embodiment the bacterium provided in the method according to the invention contains in its chromosome a polynucleotide encoding an amino acid sequence of a polypeptide shown in SEQ ID NO:4 comprising the nucleotide sequence of positions 923 to 2815 of SEQ ID NO:3.

Further in a preferred embodiment the bacterium provided in the method according to the invention contains in its chromosome a polynucleotide encoding an amino acid sequence of a polypeptide shown in SEQ ID NO:4 comprising the nucleotide sequence of positions 585 to 2815 of SEQ ID NO:3.

Further in a preferred embodiment the bacterium provided in the method according to the invention contains in its chromosome a polynucleotide encoding an amino acid sequence of a polypeptide shown in SEQ ID NO:4 comprising the nucleotide sequence of positions 565 to 2815 of SEQ ID NO:3.

Following the descriptions in the art the polypeptide shown in SEQ ID NO:4 or a processed derivative thereof. e.g. after cleaving off the signal peptide, is a mycomembrane protein and has the function or activity resp. of a cell wall-associated hydrolase, cell wall hydrolase or hydrolase.

In a further embodiment the bacterium provided in the method according to the invention comprises, in addition to the polynucleotide encoding the polypeptide shown in SEQ ID NO:4, a polynucleotide encoding a polypeptide identified by NCgl2986, wherein the amino acid proline at position 265 of the amino acid sequence is substituted by a different amino acid, preferably by Leucine.

The amino acid sequence of the polypeptide identified by NCgl2986 is also shown under SEQ ID NO:8 of the sequence listing. The nucleotide sequence encoding said polypeptide is shown under SEQ ID NO:7 of the sequence listing.

In the art the function of polypeptide identified by NCgl2986 is described as a N-acetylmuramoyl-L-alanine amidase.

The term L-lysine, where mentioned herein, in particular in the context of product formation, also comprises their ionic forms and salts, for example L-lysine mono hydrochloride or L-lysine sulfate.

For practicing the present invention bacteria of the species Corynebacterium glutamicum are used. A description of the genus Corynebacterium and the species Corynebacterium glutamicum comprised by this genus can be found in the article “Corynebacterium” by K. A. Bernard and G. Funke in Bergey's Manual of Systematics of Archaea and Bacteria (Bergey's Manual Trust, 2012).

Suitable bacteria for the method of this invention are L-lysine excreting strains of Corynebacterium glutamicum, for example L-lysine excreting strains obtained by one or several steps of strain development from strain ATCC13032 and the like and modified as described in this invention.

Strain ATCC13032 (also available as DSM20300) is the taxonomic type strain of the species Corynebacterium glutamicum. A taxonomic study of this group of bacteria based on DNA-DNA hybridization was done by Liebl et al. (International Journal of Systematic Bacteriology 41(2), 255-260, 1991). A comparative analysis of various strains of the species Corynebacterium glutamicum based on genome sequence analysis was provided by Yang and Yang (BMC Genomics 18(1): 940, 2017).

A multitude of L-lysine excreting strains of the genus Corynebacterium, in particular of the species Corynebacterium glutamicum were obtained in the art during the past decades starting from strains such as ATCC13032 and the like. They were obtained as a result of strain development programs using inter alia methods like classical mutagenesis, selection for antimetabolite resistance as well as amplification and promotor modification of genes of the biosynthetic pathway of the L-lysine by genetic engineering methods. Summaries may be found in L. Eggeling and M. Bott (Handbook of Corynebacterium glutamicum, CRC Press, 2005) or H. Yukawa and M. Inui (Corynebacterium glutamicum Biology and Biotechnology, Springer Verlag, 2013) or A. Yokota and M. Ikeda (Amino Acid Fermentation, Springer Verlag, 2017).

L-lysine excreting strains of the species Corynebacterium glutamicum are widely known in the art and can be modified as described in the present invention. For example Blombach et al. (Applied and Environmental Microbiology 75(2), 419-427, 2009) describe strain DM1933, which is deposited under accession DSM25442 according to the Budapest treaty. Strain DM1933 was obtained from ATCC13032 by several steps of strain development. Furthermore L-lysine excreting Corynebacterium glutamicum strain DM2031, deposited according to the Budapest Treaty as DSM32514 may be used. Strain DM2031 is a further developed derivative of DM1933 having enhanced L-lysine excretion ability. Other L-lysine excreting Corynebacterium glutamicum strains are e.g. described in WO2008033001 A1 and EP0841395 A1.

L-lysine excreting strains of the species Corynebacterium glutamicum typically contain a polynucleotide coding for a feedback resistant aspartokinase polypeptide variant. A feedback resistant aspartokinase polypeptide variant means an aspartokinase which is less sensitive, or desensitized resp., to inhibition by mixtures of L-lysine and L-threonine, e.g. 10 mM each, or mixtures of the L-lysine analogue S-(2-aminoethyl)-L-cysteine and L-threonine, e.g. 50 mM S-(2-aminoethyl)-L-cysteine and 10 mM L-threonine, when compared to the wild form of the enzyme, which is contained in wild strains like for example ATCC13032, ATCC14067 and ATCC13869. The EC number for aspartokinase is EC 2.7.2.4. Descriptions of polynucleotides of Corynebacterium glutamicum encoding a feedback resistant aspartokinase polypeptide variant are for example given in U.S. Pat. Nos. 5,688,671, 6,844,176 and 6,893,848. A summarizing list can be found inter alia in WO2009141330. The symbol used in the art for a gene coding for an aspartokinase polypeptide is lysC. In case the gene codes for a feedback resistant polypeptide variant the art typically uses symbols like lysC^{fbr} with fbr indicating feedback resistance.

Accordingly, said L-lysine excreting strains of the species Corynebacterium glutamicum modified as described in the present invention preferably contain at least (≥) one copy of a polynucleotide coding for a feedback resistant aspartokinase polypeptide.

SEQ ID NO:5 shows the nucleotide sequence of the coding sequence of the aspartokinase polypeptide of strain ATCC13032 and SEQ ID NO:6 the amino acid sequence of the encoded polypeptide. It is known in the art (U.S. Pat. No. 6,893,848) that exchange of the amino acid Thr at position 311 of SEQ ID NO:6 for lie imparts the enzyme feedback resistance to inhibition by mixtures of L-lysine and L-threonine.

Accordingly, it is preferred that the amino acid sequence of said feedback resistant aspartokinase polypeptide comprises the amino acid sequence of SEQ ID NO:6 containing isoleucine at position 311.

Said amino exchange can be achieved by exchanging the nucleobase cytosine (c) at position 932 of SEQ ID NO:5 to give thymine (t) (c→t substitution at the second position of the codon). The acc codon for threonine is thus altered to the atc codon for isoleucine.

It is further known in the art that exchange of the gtg start codon of the coding sequence for the aspartokinase polypeptide for atg enhances expression of the polypeptide (e.g. EP2796555 A2).

Accordingly, it is preferred that the sequence coding for a feedback resistant aspartokinase polypeptide begins with an atg start codon.

Summaries concerning the breeding of L-lysine excreting strains of Corynebacterium glutamicum may be found inter alia in L. Eggeling and M. Bott (Handbook of Corynebacterium glutamicum, CRC Press, 2005), V. F. Wendisch (Amino Acid Biosynthesis—Pathways. Regulation and Metabolic Engineering, Springer Verlag, 2007), H. Yukawa and M. Inui (Corynebacterium glutamicum Biology and Biotechnology, Springer Verlag, 2013), and Eggeling and Bott (Applied Microbiology and Biotechnology 99 (9), 3387-3394, 2015).

The term DSM denotes the depository Deutsche Sammlung für Mikroorganismen und Zellkulturen located in Braunschweig, Germany. The term ATCC denotes the depository American Type Culture Collection located in Manassas, Va., US.

Details regarding the biochemistry and chemical structure of polynucleotides and polypeptides as present in living things such as bacteria like Corynebacterium glutamicum or Escherichia coli, for example, can be found inter alia in the textbook “Biochemie” by Berg et al. (Spektrum Akademischer Verlag Heidelberg, Berlin, Germany, 2003: ISBN 3-8274-1303-6).

For sequence analysis of polynucleotides and polypeptides, e.g. sequence alignments the Clustal W program (Larkin et al.: Clustal W and Clustal X version 2.0. In: Bioinformatics 23, 2947-2948 (2007)) or public or commercially available software such as the CLC Genomics Workbench (Qiagen, Hilden, Germany) or the program MUSCLE provided by the European Bioinformatics Institute (EMBL-EBI, Hinxton, UK) may be used. For translation of the genetic code codon table 11 provided by the NCBI was used.

Corynebacterium glutamicum, in particular strain ATCC13032 and L-lysine excreting strains obtained therefrom during a strain development program, contain in their chromosome a gene, in particular one gene, encoding a polypeptide having the activity of a cell wall associated hydrolase, also referred to as cell wall hydrolase herewith, and comprising the amino acid sequence of SEQ ID NO:2. The coding sequence is shown in SEQ ID NO:1 positions 923 to 2812. The coding sequence may contain silent mutations which do not alter the amino acid sequence of the polypeptide. This context is also known as degeneracy of the genetic code in the art. Information concerning the promoter of the gene as published by Pfeifer-Sancar et al. under the old_locus_tag cg_1735 is shown in SEQ ID NO:1 (and SEQ ID NO:3).

During the work for the present invention it was found that modifying L-lysine excreting bacteria of the species Corynebacterium glutamicum by exchanging, in the encoded amino acid sequence of the polypeptide shown in SEQ ID NO:2, the amino acid valine at position 90 to isoleucine, the amino acid glycine at position 159 to arginine, the amino acid glycine at position 449 to aspartic acid and the amino acid serine at position 454 to phenylalanine increased their ability to excrete L-lysine in a fermentative process as compared to the unmodified bacterium.

During the work for the present invention it was found that additionally modifying said bacteria containing said amino acid substitutions at positions 90, 159, 449 and 454 of SEQ ID NO:2 by substituting the amino acid proline at position 265 of the amino acid sequence of the polypeptide shown in SEQ ID NO:8 by a different amino acid, preferably leucine, further increased the ability of said bacterium to excrete 1-lysine.

Further it is possible to substitute the amino acid serine at position 590 of SEQ ID NO:2 with phenylalanine in addition to the aforementioned amino acid substitutions. Still further it is obvious to try other amino acid substitutions at the indicated positions of SEQ ID NO:2 and to study the effect on L-lysine excretion of an appropriate Corynebacterium glutamicum strain.

During the work for the present invention SEQ ID NO:2 and SEQ ID NO:4 were analyzed for presence and location of signal peptide cleavage sites using SignalP software (DTU Bioinformatics, Department of Bio and Health Informatics, Lyngby, Denmark). As a result of this study the amino acid sequence between positions 49 and 50 was identified as a cleavage site and accordingly the amino acid sequence from position 1 to 49 of SEQ ID NO:2 or SEQ ID NO:4 was identified as a signal peptide for membrane translocation. It is obvious that the positions of the amino acid substitutions in the polypeptide thus processed change accordingly.

The skilled partisan is aware of a number of methods of mutagenesis how to achieve said modification(s) in the Corynebacterium glutamicum.

A mutant bacterium according to the invention can be obtained by classical in vivo mutagenesis executed with cell populations of strains of Corynebacterium glutamicum using mutagenic substances, e.g. N-methyl-N′-nitro-N-nitrosoguanidine, or ultraviolet light.

The nucleotide sequence comprising the site(s) of mutagenesis can be amplified by PCR using primers selected from SEQ ID NO: 1 or SEQ ID NO:3. By sequencing the PCR product the desired mutants are identified. Details concerning this approach can be found inter alia in U.S. Pat. No. 7,754,446. Real-time PCR in combination with FRET hybridization probes may also be used for mutation detection. The term FRET is the abbreviation for fluorescence resonance energy transfer. Cyril D S Mamotte (The Clinical Biochemist Reviews 27, 63-75 (2006)) reviews the identification of single nucleotide substitutions using this method. Further summaries concerning this method may be found in the textbook Lewin's Genes XII by Jocelyn E. Krebs, Elliott S. Goldstein and Stephan T. Kilpatrick (Jones and Bartlett Publishers, US, 2018) or elsewhere in the art.

Another common method of mutating genes of Corynebacterium glutamicum is the method of gene replacement described by Schwarzer and Pühler (Bio/Technology 9, 84-87 (1991)) and further elaborated by Schafer et al. (Gene 145, 69-73 (1994)).

Peters-Wendisch et al. (Microbiology 144, 915-927 (1998)) used the gene replacement method to inactivate the pyc gene of Corynebacterium glutamicum encoding pyruvate carboxylase. In U.S. Pat. No. 7,585,650 the method was applied to the zwf gene to realize an amino acid exchange at position 321 of the amino acid sequence of the Zwf sub-unit of the glucose 6-phosphate dehydrogenase. In U.S. Pat. No. 7,754,446 the method was applied to the rel gene to realize an amino acid exchange at position 38 of the amino acid sequence of the GTP-pyrophosphate kinase polypeptide.

In the gene replacement method, a mutation, for example, a deletion, insertion or substitution of at least one nucleobase or of one or more nucleobases resp., is provided by an isolated polynucleotide comprising the nucleotide sequence of the gene in question or a part thereof containing the mutation.

In the context of the present invention the nucleotide sequence of the gene in question is the nucleotide sequence shown under SEQ ID NO: 1 positions 923 to 2812 of the sequence listing encoding valine at position 90, glycine at position 159, glycine at position 449 and serine at position 454 of the amino acid sequence (see SEQ ID NO:2).

In the context of the present invention a preferred version of the mutation comprises the following nucleobase substitutions:
in the gtc codon (shown in positions 1190 to 1192 of SEQ ID NO: 1) coding for the amino acid valine at position 90 of SEQ ID NO:2 the nucleobase guanine is substituted for adenine to give the atc codon (shown in positions 1190 to 1192 of SEQ ID NO:3) for isoleucine at position 90 (g→a substitution at position 1190 of SEQ ID NO: 1).
in the ggg codon (shown in positions 1397 to 1399 of SEQ ID NO: 1) coding for the amino acid glycine at position 159 of SEQ ID NO:2 the nucleobase guanine at position 1397 of SEQ ID NO:1 is substituted for adenine to give the agg codon (shown in positions 1397 to 1399 of SEQ ID NO:3) for arginine at position 159 (g→a substitution at position 1397 of SEQ ID NO: 1),
in the ggt codon (shown in positions 2267 to 2269 of SEQ ID NO: 1) coding for the amino acid glycine at position 449 of SEQ ID NO:2 the nucleobase guanine at position 2268 of SEQ ID NO: 1 is substituted for adenine to give the gat codon (shown in positions 2267 to 2269 of SEQ ID NO:3) for aspartic acid at position 449 (→a substitution at position 2268 of SEQ ID NO:1), and
in the tcc codon (shown in positions 2282 to 2284 of SEQ ID NO: 1) coding for the amino acid serine at position 454 of SEQ ID NO:2 the nucleobase cytosine at position 2283 of SEQ ID NO:1 is substituted for thymine to give the ttc codon (shown in positions 2282 to 2284 of SEQ ID NO:3) for phenylalanine at position 454 (c→t substitution at position 2283 of SEQ ID NO: 1).

As a result of the mutation comprising said nucleobase substitutions the altered coding sequence shown in SEQ ID NO:3 positions 923 to 2812 is obtained encoding isoleucine at position 90, arginine at position 159, aspartic acid at position 446 and phenylalanine at position 454 of the amino acid sequence (see also SEQ ID NO:4).

The degeneracy of the genetic code is well known in the art. It is known that
the codons for isoleucine are ata, att and atc,
the codons for arginine are cgt, cgc, cga and cgg,
the codons for aspartic acid are gat and gac, and that
the codons for phenylalanine are ttt and ttc.

Accordingly, the skilled artisan is aware how to apply other nucleobase substitutions in order to obtain the amino acid exchanges at positions 90, 159, 449 and 454 of SEQ ID NO:2.

In general terms a mutated nucleotide sequence of the gene in question or a part thereof containing the mutation comprises i) a nucleotide sequence at the 5′-end of the site of mutation, which is also referred to as 5′-flanking sequence or upstream sequence in the art, ii) a nucleotide sequence at the 3′-end of the site of mutation, which is also referred to as 3′-flanking sequence or downstream sequence in the art, and iii) the nucleotide sequence of the site of mutation between i) and ii).

Said 5′-flanking sequence and 3′-flanking sequence required for homologous recombination typically have a length of at least 200 bp, at least 400 bp, at least 600 bp or at least 800 bp. The maximum length typically is 1000 bp, 1500 bp or 2000 bp.

In the case of the present invention the mutation comprises nucleobase substitutions in four codons of the coding sequence of SEQ ID NO:1 distributed between positions 1190 to 2283. Accordingly, if flanking sequences having a length of at least 400 bp are desired the mutated nucleotide sequence comprises SEQ ID NO:3 positions 790 to 2683. If flanking sequences having a length of at least 600 bp are desired the mutated nucleotide sequence comprises SEQ ID NO:3 positions 590 to 2883. If flanking sequences having a length of at least 800 bp are desired the mutated nucleotide sequence comprises SEQ ID NO:3 positions 390 to 3083. If flanking sequences having a length of at least 1000 bp are desired the mutated nucleotide sequence comprises SEQ ID NO:3 positions 190 to 3283.

The mutated nucleotide sequence provided is cloned into a plasmid vector, e.g. pK18mobsacB described by Schafer et al. (Gene 145, 69-73 (1994)), that is not capable of autonomous replication in Corynebacterium glutamicum. Said plasmid vector comprising said mutated nucleotide sequence is subsequently transferred into the desired strain of Corynebacterium glutamicum by transformation using electroporation or conjugation. After two events of homologous recombination comprising a recombination event within the 5′-flanking sequence provided by the plasmid vector with the homologous sequence of the Corynebacterium glutamicum chromosome and a recombination event within the 3′-flanking sequence provided by the plasmid vector with the homologous sequence of the Corynebacterium glutamicum chromosome, one effecting integration and one effecting excision of said plasmid vector, the mutation is incorporated in the Corynebacterium glutamicum chromosome. Thus the nucleotide sequence of the gene in question contained in the chromosome of said desired strain is replaced by the mutated nucleotide sequence. The presence of the mutation in the desired strain is then confirmed e.g. by analysis of the nucleotide sequence or real-time PCR using FRET as described above.

An event of homologous recombination may also be referred to as crossing over.

It is preferred that the L-lysine excreting Corynebacterium glutamicum strains provided for the method of the present invention have the ability to excrete ≥0.25 g/l, preferably ≥0.5 g/l, particularly preferred ≥1.0 g/l, very particularly preferred ≥2.0 g/l of L-lysine in a suitable medium under suitable conditions.

In a fermentative process according to the invention a Corynebacterium glutamicum modified in accordance with the present invention and having the ability to excrete L-lysine is cultivated in a suitable medium under suitable conditions. Due to said ability to excrete said L-lysine the concentration of the L-lysine increases and accumulates in the medium during the fermentative process and the L-lysine is thus produced.

The fermentative process may be a discontinuous process like a batch process or a fed batch process or a continuous process. A summary concerning the general nature of fermentation processes is available in the textbook by H. Chmiel (Bioprozesstechnik, Spektrum Akademischer Verlag, 2011), in the textbook of C. Ratledge and B. Kristiansen (Basic Biotechnology, Cambridge University Press, 2006) or m the textbook of V. C. Hass and R. Pörtner (Praxis der Bioprozesstechnik Spektrum Akademischer Verlag, 2011).

A suitable medium used for the production of L-lysine by a fermentative process contains a carbon source, a nitrogen source, a phosphorus source, inorganic ions and other organic compounds as required.

Suitable carbon sources include glucose, fructose, sucrose as well as the corresponding raw materials like starch hydrolysate, molasses or high fructose corn syrup.

As nitrogen source organic nitrogen-containing compounds such as peptones, meat extract, soybean hydrolysates or urea, or inorganic compounds such as ammonium sulphate, ammonium chloride, ammonium phosphate, ammonium carbonate, ammonium nitrate, ammonium gas or aqueous ammonia can be used.

As phosphorus source, phosphoric acid, potassium dihydrogen phosphate or dipotassium hydrogen phosphate or the corresponding sodium-containing salts can be used.

Inorganic ions like potassium, sodium, magnesium, calcium, iron and further trace elements etc. are supplied as salts of sulfuric acid, phosphoric acid or hydrochloric acid.

Other organic compounds mean essential growth factors like vitamins e.g. thiamine or biotin or L-amino acids e.g. L-homoserine.

The media components may be added to the culture in form of a single batch or be fed in during the cultivation in a suitable manner.

During the fermentative process, the pH of the culture can be controlled by employing basic compounds such as sodium hydroxide, potassium hydroxide, ammonia or aqueous ammonia, or acidic compounds such as phosphoric acid or sulphuric acid in a suitable manner. The pH is generally adjusted to a value of from 6.0 to 8.5, preferably 6.5 to 8.0. To control foaming, it is possible to employ antifoam agents such as, for example, fatty acid polyglycol esters. To maintain the stability of plasmids, it is possible to add to the medium suitable selective substances such as, for example, antibiotics. The fermentative process is preferably carried out under aerobic conditions. In order to maintain these conditions, oxygen or oxygen-containing gas mixtures such as, for example air are introduced into the culture. The fermentative process is carried out, where appropriate, at elevated pressure, for example at an elevated pressure of 0.03 to 0.2 MPa. The temperature of the culture is normally from 25° C. to 40° C., preferably from 30° C. to 37° C. In a discontinuous process, the cultivation is continued until an amount of the L-lysine sufficient for being recovered has been formed. The cultivation is then completed. This aim is normally achieved within 10 hours to 160 hours. In continuous processes, longer cultivation times are possible.

Examples of suitable media and culture conditions can be found inter alia in L. Eggeling and M. Bott (Handbook of Corynebacterium glutamicum, CRC Press, 2005) and the patent documents U.S. Pat. Nos. 5,770,409, 5,990,350, 5,275,940, 5,763,230 and 6,025,169.

Thus the fermentative process results in a fermentation broth which contains the desired L-lysine.

A product containing the L-lysine is then recovered or manufactured in liquid or solid from the fermentation broth.

A “fermentation broth” means a medium in which a Corynebacterium glutamicum described in the invention has been cultivated for a certain time and under certain conditions.

When the fermentative process is completed, the resulting fermentation broth accordingly comprises:
a) the biomass (cell mass) of the Corynebacterium glutamicum of the invention, said biomass having been produced due to propagation of the cells of said Corynebacterium glutamicum,
b) the desired L-lysine accumulated during the fermentative process,
c) the organic by-products accumulated during the fermentative process, and
d) the components of the medium employed which have not been consumed in the fermentative process.

The organic by-products include compounds which may be formed by the Corynebacterium glutamicum of the invention during the fermentative process in addition to production of the L-lysine.

The fermentation broth is removed from the culture vessel or fermentation tank, collected where appropriate, and used for providing a product containing the L-lysine, in liquid or solid form. The expression “recovering the L-lysine-containing product” is also used for this. In the simplest case, the L-lysine-containing fermentation broth itself, which has been removed from the fermentation tank, constitutes the recovered product.

An abundance of technical instructions for measures a), b), c) and d) are available in the art.

Removal of water (measure a)) can be achieved inter alia by evaporation, using e.g. a falling film evaporator, by reverse osmosis or nanofiltration. The concentrates thus obtained can be further worked up by spray drying or spray granulation. It is likewise possible to dry the fermentation broth directly using spray drying or spray granulation.

Accordingly, a method according to the invention comprises extracting or substantially eliminating water from said fermentation broth. In particular at least 40% (w/w), preferred at least 90% (w/w), more preferred at least 95% (w/w) water are extracted from the fermentation broth.

Removal of the biomass (measure b)) can be achieved inter alia by centrifugation, filtration or decantation or a combination thereof.

Removal of the organic by-products (measure c)) or removal of residual components of the medium (measure d) can be achieved inter alia by chromatography, e.g. ion exchange chromatography, treatment with activated carbon or crystallization. In case the organic by-products or residual components of the medium are present in the fermentation broth as solids they can be removed by measure b).

Accordingly, the manufacturing of an L-lysine product according to the invention comprises a purification step, preferably selected from the group consisting ion exchange chromatography, treatment with activated carbon or crystallization.

Thus e. g. a product containing L-lysine×HCl, preferably containing ≥80% L-lysine×HCl, particularly preferred ≥90% L-lysine×HCl or ≥95% L-lysine×HCl can be obtained.

General instructions on separation, purification and granulation methods can be found inter alia in the book of R. Ghosh “Principles of Bioseparation Engineering” (World Scientific Publishing, 2006), the book of F. J. Dechow “Separation and Purification Techniques in Biotechnology” (Noyes Publications, 1989), the article “Bioseparation” of Shaeiwitz et al. (Ullmann's Encyclopedia of Industrial Chemistry, Wiley-VCH, 2012) and the book of P. Semo et al. “Granulieren” (Editio Cantor Verlag, 2007).

A downstream processing scheme for L-lysine products can be found in the article “L-lysine Production” of R. Kelle et al. (L. Eggeling and M. Bott (Handbook of Corynebacterium glutamicum, CRC Press, 2005)). U.S. Pat. No. 5,279,744 teaches the manufacturing of a purified L-lysine product by ion exchange chromatography. U.S. Pat. No. 5,431,933 teaches the manufacturing of dry L-amino acid products, e. g. an L-lysine product, containing most of the constituents of the fermentation broth.

Thus a concentration or purification of the L-lysine is achieved and a product having the desired content of said L-lysine is provided.

Analysis of L-lysine to determine its concentration at one or more time(s) during the fermentation can take place by separating the L-lysine by means of ion exchange chromatography, preferably cation exchange chromatography, with subsequent post-column derivatization using ninhydrin, as described in Spackman et al. (Analytical Chemistry 30: 1190-1206 (1958)). It is also possible to employ ortho-phthalaldehyde rather than ninhydrin for post-column derivatization. An overview article on ion exchange chromatography can be found in Pickering (LC.GC (Magazine of Chromatographic Science 7(6):484-487 (1989)). It is likewise possible to carry out a pre-column derivatization, for example using ortho-phthalaldehyde or phenyl isothiocyanate, and to fractionate the resulting amino acid derivates by reversed-phase chromatography (RP), preferably in the form of high-performance liquid chromatography (HPLC). A method of this type is described, for example, in Lindroth et al. (Analytical Chemistry 51:1167-1174 (1979)). Detection is carried out photometrically (absorption, fluorescence). A review regarding amino acid analysis can be found inter alia in the textbook “Bioanalytik” by Lottspeich and Zorbas (Spektrum Akademischer Verlag, Heidelberg, Germany 1998).

### EXPERIMENTAL SECTION

### A) Materials and Methods

The molecular biology kits, primers and chemicals used, and some details of the methods applied are briefly described herewith.

### 1. Antibiotics and Chemicals

a. Kanamycin: Kanamycin solution from Streptomyces kanamyceticus from Sigma Aldrich (St. Louis, USA, Cat. no. K0254).
b. Nalidixic acid: Nalidixic acid sodium salt from Sigma Aldrich (St. Louis, USA, Cat. no. N4382).
c. If not stated otherwise, all chemicals were purchased analytically pure from Merck (Darmstadt, Germany), Sigma Aldrich (St. Louis, USA) or Carl-Roth (Karlsruhe, Germany).

### 2. Cultivation

If not stated otherwise, all cultivation/incubation procedures were performed as follows herewith:

a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Einsbach, Germany) at 37° C. and 200 rpm.
b. LB agar (MILLER) from Merck (Darmstadt, Germany Cat. no. 110283) was used for cultivation of E. coli strains on agar plates. The agar plates were incubated at 37° C. in an INCU-Line® mini incubator from VWR (Radnor, USA).
c. Brain heart infusion broth (BHI) from Merck (Darmstadt, Germany: Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Einsbach, Germany) at 33° C. and 200 rpm.
d. Brain heart agar (BHI-agar) from Merck (Darmstadt, Germany; Cat. no. 113825) was used for cultivation of C. glutamicum strains on agar plates. The agar plates were incubated at 33° C. in an incubator from Heraeus Instruments with Kelvitron® temperature controller (Hanau, Germany).

### 3. Determining Optical Density

a. The optical density of bacterial suspensions in shake flask cultures was determined at 600 nm (OD600) using the BioPhotometer from Eppendorf AG (Hamburg, Germany).
b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).

### 4. Centrifugation

a. Benchtop centrifuge for reaction tubes with a volume up to 2 ml

Bacterial suspensions with a maximum volume of 2 ml were caused to sediment using 1 ml or 2 ml reaction tubes (e.g. Eppendorf Tubest 3810X) using an Eppendorf 5417 R centrifuge (5 min. at 13.000 rpm).

b. Benchtop centrifuge for tubes with a volume up to 50 ml

Bacterial suspensions with a maximum volume of 50 ml were caused to sediment using 15 ml or 50 ml centrifuge tubes (e.g. Falcon™ 50 ml Conical Centrifuge Tubes) using an Eppendorf 5810 R centrifuge for 10 min. at 4.000 rpm.

### 5. Polymerase Chain Reaction (PCR)

PCR with a proofreading (high fidelity) polymerase was used to amplify a desired segment of DNA before Sanger sequencing.

a. The Phusion, High-Fidelity DNA Polymerase Kit (NEB Phusion Kit) from New England BioLabs Inc. (Ipswich, USA; Cat. No. M0530) was used for template-correct amplification of selected DNA regions according to the instructions of the manufacturer (see table 1).

TABLE 1
Thermocycling conditions for PCR with Phusion ®
High-Fidelity DNA Polymerase Kit from NEB Inc.
PCR-program

| | Time | T | |
|---|---|---|---|
| Step | [min.] | [° C.] | Description |
| 1 | 00:30 | 98 | Initial denaturation step |
| 2 | 00:05 | 98 | Denaturation step |
| 3 | 00:30 | 60 | Annealing step |
| 4 | 00:xx | 72 | Elongation step |
| | | | 15-30 sec. per kb DNA |
| | | | Repeat step 2 to 4: 30 x |
| 5 | 05:00 | 72 | Final Elongation step |
| 6 | Hold | 4 | Cooling step |

b. Primer

The oligonucleotides used were synthesized by eurofins genomics GmbH (Ebersberg, Germany) using the phosphoramidite method described by McBride and Caruthers (Tetrahedron Lett. 24, 245-248, 1983).

c. Template

As PCR template either a suitably diluted solution of isolated plasmid DNA or of isolated total DNA from a C. glutamicum liquid culture or the total DNA contained in a colony was used (colony PCR). For said colony PCR the template was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogeräte GmbH (Sundem, Germany) and then the PCR reagents were added to the template in the PCR reaction tube.

d. PCR Cycler

PCRs were carried out in PCR Cyclers type Mastercycler or Mastercycler nexus gradient from Eppendorf AG (Hamburg, Germany).

### 6. Detection of Mutations Using FRET

The presence of a given mutation, e.g. a nucleobase exchange, was detected by real-time PCR in combination with FRET hybridization probes. The term FRET is the abbreviation for fluorescence resonance energy transfer. As real-time PCR instrument a Lightcycler from Roche Diagnostics® was used (see below).

This method was e. g. used by M. J. Lay and C. T. Wittwer (Clinical Chemistry 42 (12), 2262-2267 (1997)) for the genotyping of factor V Leiden. Cyril D S Mamotte (The Clinical Biochemist Reviews 27, 63-75 (2006) reviews the genotyping of single nucleotide substitutions using this method. Summaries concerning this method may be found in the textbooks Lewin's Genes XII by Jocelyn E. Krebs, Elliott S. Goldstein and Stephan T. Kilpatrick (Jones and Bartlett Publishers. US, 2018), Molecular Diagnostics, 12 Tests that changed everything by W. Edward Highsmith (Humana Press, Springer, New York, 2014) or elsewhere in the art.

The FRET hybridization donor probe was labelled with the fluorescent dye fluorescein and the acceptor probe with the fluorescent dye LC-Red640. In essence the detection method comprised three steps: colony PCR, probe hybridization, and subsequent melting curve analysis. The method is simply referred to as real-time PCR herewith.

a. Primers and Probes

The oligonucleotides used were synthesized by eurofins genomics GmbH (Ebersberg, Germany).

b. Template

As PCR template the total DNA contained in a colony was used. It was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogeräte GmbH (Sundem, Germany) and then the PCR reagents were added to the template in the PCR reaction tube.

e. Reaction Mix

The Type-it® Fast SNP probe PCR Kit (Type-it Kit) from Qiagen (Hilden, Germany, Cat. No. 206045) was used for real-time detection of the mutations. Therefore 2.5 μl of the Qiagen Fast SNP Puffer (2×) was mixed with 0.5 μl of each of the LC-PCR-Primers [10 μM] and 0.5 μl of each of the 1:500) diluted acceptor and donor probe [100 pmol/μl] to get the mastermix for the real-time PCR.

TABLE 2
Thermocycling conditions for PCR with the LightCycler ®
(step 1-3) and melting curve analysis (step 4-6).
PCR-program

| | Time | T | |
|---|---|---|---|
| Step | [sec.] | [° C.] | Description |
| 1 | 15 | 95 | Denaturation step (and Activation of |
| | | | HotStarTaq ™ DNA polymerase) |
| 2 | 05 | 55 | Annealing step |
| 3 | 30 | 72 | Elongation step |
| | | | Repeat step 1 to 3: 50 x |
| 4 | 10 | 95 | Denaturation step |
| 5 | 30 | 40 | Probe hybridization |
| 6 | | 40-80 | Melting curve analysis |
| 7 | | 80-40 | Cooling |

f. PCR Cycler

The reactions were carried out in a LightCycler® 2.0 Instrument and analyzed with LightCycler® Software 4.1 of Roche Diagnostics (Rotkreuz, Switzerland).

7. Chemical Transformation of E. coli

E. coli K-12 strain S17-1 was used as donor for conjugational transfer of plasmids based on pK18mobsacB from E. coli to C. glutamicum. Strain S17-1 is described by Simon, R et al. (Bio/Technology 1, 784-794, 1983). It is available from the American Type Culture Collection under the access number ATCC47055.

Chemically competent E. coli S17-1 cells were made as follows: A preculture of 10 ml LB medium (10 ml liquid medium per 100 ml Erlenmeyer flask with 3 baffles) was inoculated with 100 μl bacterial suspension of strain S17-1 and the culture was incubated overnight for about 18 h at 37° C. and 250 rpm. The main culture (70 ml LB contained in a 250 ml Erlenmeyer flask with 3 baffles) was inoculated with 300 μl of the preculture and incubated up to an OD600 of 0.5-0.8 at 37° C. The culture was centrifuged for 6 min. at 4° C. and 4000 rpm and the supernatant was discarded. The cell pellet was resuspended in 20 ml sterile, ice-cold 50 mM CaCl₂ solution and incubated on ice for 30 min. After another centrifugation step, the pellet was resuspended in 5 ml ice-cold 50 mM CaCl₂ solution and the suspension incubated on ice for 30 min. The cell suspension was then adjusted to a final concentration of 20% glycerol (v/v) with 85% (v/v) sterile ice-cold glycerol. The suspension was divided into 50 μl aliquots and stored at −80° C.

To transform S17-1 cells, the protocol according to Tang et al. (Nucleic Acids Res. 22(14), 2857-2858, 1994) with a heat shock of 45 sec. was used.

8. Conjugation of C. glutamicum

The pK18mobsacB plasmid system described by Schafer et al. (Gene 145, 69-73, 1994) was used to integrate desired DNA fragments into the chromosome of C. glutamicum. A modified conjugation method of Schafer et al. (Journal of Bacteriology 172, 1663-1666, 1990) was used to transfer the respective plasmid into the desired C. glutamicum recipient strain.

Liquid cultures of the C. glutamicum strains were carried out in BHI medium at 33° C. The heat shock was carried out at 48.5° C. for 9 min. Transconjugants were selected by plating the conjugation batch on EM8 agar (Table 3), which was supplemented with 25 mg/l kanamycin and 50 mg/l nalidixic acid. The EM8 agar plates were incubated for 72 h at 33° C.

TABLE 3
Composition of the EM8 agar

| Components | Concentration (g/l) |
|---|---|
| Glucose (sterile-filtered) | 23 |
| CSL (corn steep liquor; Roquette; solid | 30 |
| content 48 ± 2% w/w) | |
| Peptone from soymeal (Merck, Germany) | 40 |
| (NH₄)₂SO₄ | 8 |
| Urea | 3 |
| KH₂PO₄ | 4 |
| MgSO₄ •7 H₂O | 0.5 |
| FeSO₄ •7 H₂O | 0.01 |
| CuSO₄•5 H₂O | 0.001 |
| ZnSO₄•7 H₂O | 0.01 |
| Calcium pantothenate, D(+) | 0.01 |
| Thiamine | 0.001 |
| Inositol | 0.1 |
| Nicotinic acid | 0.001 |
| Biotin (sterile-filtered) | 0.005 |
| CaCO₃ (autoclaved separately) | 1.6 |
| Agar-Agar (Merck, Germany) | 14 |

Sterile toothpicks were used to transfer the transconjugants onto BHI agar, which was supplemented with 25 mg/l kanamycin and 50 mg/l nalidixic acid. The agar plates were incubated for 20 h at 33° C. The cultures of the respective transconjugants produced in this manner were then propagated further for 24 h at 33° C. in 10 ml BHI medium contained in 100 ml Erlenmeyer flasks with 3 baffles. An aliquot was taken from the liquid culture suitably diluted and plated (typically 100 to 200 μl) on BHI agar which was supplemented with 10% saccharose. The agar plates were incubated for 48 h at 33° C. The colonies growing on the saccharose containing agar plates were then examined for the phenotype kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined for integration of the desired DNA fragment by means of real-time PCR.

### 9. Determining Nucleotide Sequences

Nucleotide sequences of DNA molecules were determined by eurofins genomics GmbH (Ebersberg, Germany) by cycle sequencing, using the dideoxy chain termination method of Sanger et al. (Proceedings of the National Academy of Sciences USA 74, 5463-5467, 1977), on Applied Biosystems® (Carlsbad, Calif., USA) 3730xl DNA Analyzers. Clonemanager Professional 9 software from Scientific & Educational Software (Denver, USA) was used to visualize and evaluate the sequences.

10. Glycerol Stocks of E. coli and C. glutamicum Strains

For long time storage of E. coli- and C. glutamicum strains glycerol stocks were prepared. Selected E. coli clones were cultivated in 10 ml LB medium supplemented with 2 g/l glucose. Selected C. glutamicum clones were cultivated in two-fold concentrated BHI medium supplemented with 2 g/l glucose. Cultures of plasmid containing E. coli strains were supplemented with 50 mg/l kanamycin. Cultures of plasmid containing C. glutamicum strains were supplemented with 25 mg/l kanamycin. The medium was contained in 100 ml Erlenmeyer flasks with 3 baffles. It was inoculated with a loop of cells taken from a colony and the culture incubated for about 18 h at 37° C. and 200 rpm in the case of E. coli and 33° C. and 200) rpm in the case of C. glutamicum. After said incubation period 1.2 ml 85% (v/v) sterile glycerol were added to the culture. The obtained glycerol containing cell suspension was then aliquoted in 2 ml portions and stored at −80° C.

### 11. Cultivation System According to Wouter Duetz (WDS)

The milliliter-scale cultivation system according to Duetz (Trends Microbiol. 2007; 15(10):469-75) was used to investigate the performance of the C. glutamicum strains constructed. For this purpose, 24-deepwell microplates (24 well WDS plates) from EnzyScreen BV (Heemstede. Netherlands; Cat. no. CR1424), filled with 2.5 mL medium were used.

Precultures of the strains were done in 10 ml two-fold concentrated BHI medium. The medium was contained in a 100 ml Erlenmeyer flask with 3 baffles. It was inoculated with 100 μl of a glycerol stock culture and the culture incubated for 24 h at 33° C. and 200 rpm. After said incubation period the optical densities OD600 of the precultures were determined.

The main cultures were done by inoculating the 2.5 ml medium containing wells of the 24 Well WDS-Plate with an aliquot of the preculture to give an optical density OD600 of 0.1. As medium for the main culture CGXII medium described by Keilhauer et al. (J. Bacteriol. 1993 September; 175(17); 5595-5603) was used. For convenience the composition of the CGXII medium is shown in table 4.

TABLE 4
Composition of Keilhauer's CGXII medium.

| Components | Concentration (g/l) |
|---|---|
| MOPS (3-(N-Morpholino)propanesulfonic acid) | 42 |
| (NH₄)₂SO₄ | 20 |
| Urea | 5 |
| KH₂PO₄ | 1 |
| K₂HPO₄ | 1 |
| MgSO₄•7 H₂O | 0.25 |
| CaCl₂ | 0.01 |
| FeSO₄•7 H₂O | 0.01 |
| MnSO₄ H₂O | 0.01 |
| ZnSO₄ •7 H₂O | 0.001 |
| CuSO₄•5 H₂O | 0.0002 |
| NiCl₂ 6 H₂O | 0.00002 |
| Biotin (sterile-filtered) | 0.0002 |
| Protocatechuic acid (sterile-filtered) | 0.03 |
| Carbon source (sterile-filtered) | as needed |
| adjust the pH to 7 with NaOH | |

These main cultures were incubated for approximately 45 h at 33° C. and 300 rpm in an Infors HT Multitron standard incubator shaker from Infors GmbH (Bottmingen. Switzerland) until complete consumption of glucose.

The glucose concentration in the suspension was analyzed with the blood glucose-meter OneTouch Vita® from LifeScan (Johnson & Johnson Medical GmbH, Neuss, Germany). After cultivation the culture suspensions were transferred to a deep well microplate. A part of the culture suspension was suitably diluted to measure the OD600. Another part of the culture was centrifuged and the concentration of L-amino acids, in particular L-lysine, and residual glucose were analyzed in the supernatant.

### 12. Amino Acid Analyzer

The concentration of L-lysine and other L-amino acids, e.g. L-valine, in the culture supernatants was determined by ion exchange chromatography using a SYKAM S433 amino acid analyzer from SYKAM Vertriebs GmbH (Fürstenfeldbruck, Germany). As solid phase a column with spherical, polystyrene-based cation exchanger (Peek LCA N04/Na, dimension 150×4.6 mm) from SYKAM was used. Depending on the L-amino acid the separation took place in an isocratic run using a mixture of buffers A and B for elution or by gradient elution using said buffers. As buffer A an aqueous solution containing in 20 l 263 g trisodium citrate, 120 g citric acid, 1100 ml methanol, 100 ml 37% HCl and 2 ml octanoic acid (final pH 3.5) was used. As buffer B an aqueous solution containing in 20 l 392 g trisodium citrate, 100 g boric acid and 2 ml octanoic acid (final pH 10.2) was used. The free amino acids were colored with ninhydrin through post-column derivatization and detected photometrically at 570 nm.

13. Glucose Determination with Continuous Flow System (CFS)

A SANplus multi-channel continuous flow analyzer from SKALAR analytic GmbH (Erkelenz, Germany) was used to determine the concentration of glucose in the supernatant. Glucose was detected with a coupled-enzyme assay (Hexokinase/Glucose-6-Phosphate-Dehydrogenase) via NADH formation.

### B) Experimental Results

### Example 1

Sequence of the NCgl1480* Gene of C. glutamicum Strain DM1933

The “*” in the designation NCgl1480* indicates that according to this invention the coding sequence of NCgl1480* comprises a sequence coding for 26 additional amino acids at the 5′-terminus as compared to the information given under locus_tag NCgl1480 (see SEQ ID NO: 1 and SEQ ID NO:2).

Strain DM1933 is an L-lysine producer described by Blombach et al. (Applied and Environmental Microbiology 75(2), 419-427, 2009). It was deposited according to the Budapest treaty at the DSMZ under accession number DSM25442.

The nucleotide sequence of the chromosome of strain DM1933 was determined by Illumina whole-genome sequencing technology (Illumina Inc., San Diego, Calif., US). See e.g. Benjak et al. (2015) Whole-Genome Sequencing for Comparative Genomics and De Novo Genome Assembly. In: Parish T., Roberts D. (eds) Mycobacteria Protocols. Methods in Molecular Biology, Vol 1285. Humana Press, NY, US) and Bennet, S. (Pharmacogenomics 5(4), 433-438, 2004).

It was found that the nucleotide sequence of the NCgl1480* coding sequence of strain DM1933 including the nucleotide sequence upstream and downstream thereof was identical to that of ATCC13032 shown in SEQ ID NO: 1.

DM1933 contains in its chromosome a variant of the aspartokinase gene encoding a feedback resistant aspartokinase polypeptide. Said feedback resistant aspartokinase polypeptide has the amino acid sequence of SEQ ID NO:6 of the sequence listing, wherein the amino acid threonine (Thr) at position 311 of the amino acid sequence is replaced by isoleucine (Ile). In U.S. Pat. No. 7,338,790 the abbreviation “lysC T311I” is used to indicate said exchange. Blombach et al. use the abbreviation “lysC(T311I)”.

### Example 2

Construction of Plasmid pK18mobsacB_NCgl1480*_Mut
Plasmid pK18mobsacB_NCgl1480*_Mut was constructed to enable incorporation of the mutations causing the amino acid exchanges V90I, G159R, G449D and S454F into the nucleotide sequence of the chromosome of strain DM1933. The plasmid was based on the mobilizable vector pK18mobsacB described by Schafer et al. (Gene 145, 69-73, 1994). For the construction of pK18mobsacB_NCgl1480*_Mut the NCgl1480*_Mut sequence according to SEQ ID NO: 11 was synthesized and subcloned into pK18mobsacB by GeneArt (ThermoFisher Scientific (Waltham, USA)).

To assemble the plasmid pK18mobsacB_NCgl1480*_Mut the two polynucleotides i.e. the vector pK18mobsacB cut with SbfI and the polynucleotide NCgl1480*_Mut cut with SbfI were ligated and the ligation mixture used to transform an appropriate E. coli strain (Geneart; ThermoFisher Scientific (Waltham, USA)).

DNA of plasmid pK18mobsacB_NCgl1480*_Mut was isolated from a transformant.

### Example 3

Construction of Strain DM1933_NCgl1480*_Mut The plasmid pK18mobsacB_NCgl1480*_Mut obtained in example 2 was used to incorporate the mutations leading to the amino acid exchanges V90I, G159R, G449D and S454F (see nucleotide positions 1190, 1397, 2268 and 2283 of SEQ ID NO:3) into the chromosome of the L-lysine producer DM1933.

Chemically competent cells of E. coli strain S17-1 were transformed with plasmid DNA of pK18mobsacB_NCgl1480*_Mut. The modified conjugation method of Schafer et al. (Journal of Bacteriology 172, 1663-1666, 1990) as described in materials and methods was used for conjugal transfer into the strain DM1933 and for selection of transconjugant clones by virtue of their saccharose resistance and kanamycin sensitivity phenotype.

Transconjugant clones were analyzed by two separate real-time PCRs to detect the mutations leading to the amino acid exchanges V90I and S454F using the Type-it Kit. To detect the mutation leading to the amino acid exchange V90I the primers LC-NCgl1480_1 and LC-NCgl1480_2 for PCR amplification and NCgl1480_90_C as acceptor probe and NCgl1480_90_A as donor probe for melting curve analysis were used (table 5). To detect the mutation leading to the amino acid exchange S454F the primers LC-NCgl1480_3 and LC-NCgl1480_4 for PCR amplification and NCgl1480_454_C as acceptor probe and NCgl1480_454_A as donor probe for melting curve analysis were used (table 5).

TABLE 5
List of primers and probes used for real-time PCR.

| name | sequence |
|---|---|
| LC-NCgl1480_1 | AGCGGAGCTTTGCTTAGTTC |
| LC-NCgl1480_2 | TGCTTCGATCTGCGCTTGAG |
| NCgl1480_90_C¹ | CCAGCTCGATGCGGTTAATT |
| NCgl1480_90_A² | AGGGACTTGTTCACTTCTTCACGCAGAGCACCC |
| | AT |
| LC-NCgl1480_3 | CGCTGCGGCTGCTCTAATTG |
| LC-NCgl1480_4 | GCGTACTGCACACCCAACTG |
| NCgl1480_454_C1 | GTTGCTCTGGAAATCTCCAG |
| NCgl1480_454_A² | CTGTGGAATCGTTGCCTGTGGAATCGTTGTCGC |
| | TG |

¹acceptor probe labelled with LC-Red640 at the 5′-end and phosphorylated at the 3′-end
²donor probe labelled with fluorescein at the 3′-end

One of the transconjugant clones thus characterized was called DM1933_NCgl1480*_Mut. The nucleotide sequence of the chromosomal region of strain DM1933_NCgl1480*_Mut containing the mutated nucleotide sequence was analyzed by Sanger sequencing.

For this purpose a PCR amplificate was produced spanning the site of mutations. A colony PCR was done using the primers LC-NCgl1480_1 and LC-NCgl1480_4 (see table 5) and the NEB Phusion Kit with an elongation time of 45 sec. (table 1, step 4). The amplificate obtained was then sequenced using the same primers (LC-NCgl1480_1 and LC-NCgl1480_4).

The nucleotide sequences of the primers and probes used in this context (see table 5) are also shown in SEQ ID NO: 12 to 19 of the sequence listing.

The nucleotide sequence obtained is shown in SEQ ID NO:20. The result showed that strain DM1933_NCgl1480*_Mut contained all desired mutations, or the desired mutated nucleotide sequence resp., in its chromosome with the effect of the amino acid exchanges V90I, G1591R, G449D and S454F.

A glycerol stock culture of the transconjugant clone was prepared and used as starting material for further investigations.

### Example 4

### L-Lysine Production by Strain DM1933_NCgl1480*_Mut

Strains DM1933 (reference) and DM1933_NCgl1480*_Mut obtained in example 3 were analyzed for their ability to produce L-lysine from glucose by batch cultivation using the cultivation system according to Wouter Duetz.

As medium CGXII containing 20 g/l glucose as carbon source was used. The cultures were incubated for 45 h until complete consumption of glucose as confirmed by glucose analysis using blood glucose-meter and the concentrations of L-lysine and the optical density OD660 were determined. The result of the experiment is presented in table 7.

TABLE 7
L-lysine production by strain DM1933_NCgl1480*_Mut.

| strain | L-lysine¹ (g/l) | OD660 |
|---|---|---|
| DM1933 | 3.7 | 9.5 |
| DM1933_NCgl1480*_Mut | 4.1 | 8.9 |

¹as L-lysine x HCl

The experiment shows that L-lysine production was increased in strain DMI933_NCgl1480*_Mut as compared to the parent strain DM1933.

1. A method for the fermentative production of L-lysine, comprising:
a) providing a bacterium of the species Corynebacterium glutamicum having an ability to excrete L-lysine, containing in its chromosome a polynucleotide encoding a polypeptide comprising an amino acid sequence of SEQ ID NO:4,
b) cultivating the bacterium in a suitable medium under suitable conditions, and
c) accumulating said L-lysine in the suitable medium to form an L-lysine containing fermentation broth.

2. The method as claimed in claim 1, wherein in the bacterium provided, said polynucleotide, encoding said amino acid sequence, comprises a nucleotide sequence of SEQ ID NO:3 positions 923 to 2812.

3. The method as claimed in claim 1, wherein in the bacterium provided, the polynucleotide, encoding said amino acid sequence, comprises a nucleotide sequence of SEQ ID NO:3 positions 923 to 2815.

4. The method as claimed in claim 1, wherein in the bacterium provided, the polynucleotide, encoding said amino acid sequence, comprises a nucleotide sequence of SEQ ID NO:3 positions 585 to 2815.

5. The method as claimed in claim 1, wherein in the bacterium provided, the polynucleotide, encoding said amino acid sequence, comprises a nucleotide sequence of SEQ ID NO:3 positions 565 to 2815.


SEQUENCE LISTING (20 sequences)

SEQ ID NO: 1 - DNA, 3815 bp, Corynebacterium glutamicum ATCC13032
  misc_feature (1)..(411): 3′-sequence of the cds of NCgl1481
  misc_feature (412)..(413): taa stop codon
  misc_feature (565)..(565): nucleobase cytosine
  misc_feature (585)..(585): nucleobase guanine
  misc_feature (632)..(637): taatgt motif according to Pfeifer-Sancar et al.
  misc_feature (644)..(644): transcriptional start site according to Pfeifer-Sancar et al.
  CDS (923)..(2812): coding sequence
  misc_feature (1001)..(2812): coding sequence according to NCgl1480
  misc_feature (1190)..(1192): gtc codon for valine
  misc_feature (1397)..(1399): ggg codon for glycine
  misc_feature (2267)..(2269): ggt codon for glycine
  misc_feature (2282)..(2284): tcc codon for serine
  misc_feature (2813)..(2815): tag stop codon
  misc_feature (2889)..(3815): 5′-sequence of the cds of NCgl1479
tttccgactttggaaaaagaccttattaatgttgtgacagatgcaaaggaatcagattttggatctttaggggtcgtgattcttgatgaaactccagtgatgacttccaacctgagggatattgcgcaggagctgttgattcaaacggatctggacaccgttgtggtgcgggctccaatgtcggctgcggtggtgagtgatgttcattcgagggcggcgcttgagtcagggcagcatgatttgcttgggaccactgattacgtgctgggaacggagcttttggtacaggatgtaacggaatcgacggtggggaatattgattggggtcaattgctgatttgggggttggttgctttggcaatcgccgtggttgttgcgggtgcgtctgtgcgtcgaaaagcaatatctttataagtaaagttctaaagctttacttatagcactttcggtaggtgtcgagaaaattcccaaattgcggaaaattcacatactttgtgccacaaagtgataaacatcacaaaatttcatgactcagccattcatgcaggccagaacatgcaaaaccttgcacgctctggccttttgttatgtaactgttgtgaaagatgtatcaaaagttaccaaggtgacttaatgttcagatatcgactcatgggttgttttccaaaacttgaatcaatttgagtaacagtagttatcaagcgttaaaccttgaaacttccactctttttactgatgggcatttgggcaaacggggaagcttgctggagatgcaataaatcggtgaatggttggtgcaatgaagaggagagtggcccatagtttccgctacggaacgcggagtctttacgtttcaattcttggcaagtatctagcgtcaaggcttgatatgtgaagggattcggattgaacaggagaacatgtggccaatctagattcagttcgccctggggtgcgggcagctcttcgacgtacccatacccaggtaaaaggtgtcacgttgaccagggcgttgattgcgcttgcagtaagcggagctttgcttagttccatgactccggcggtggcgcagccacagaatccggatgacgcagccattgcacaggcagaggaaaatgtttcggcgggcgatggggaagtcgcccgcctggcaggatctttgtccagcactgacgcggaaattaaccgcgtcgagctggaaatgggtgctctgcgtgaagaagtgaacaagtccctcgtggatttgcatgatgcgcaggcaatcgccgagcaggcccgccaagatgcacttgcagccaagaaggatctcgatgattctcaagcgcagatcgaagcagcccaagagcgccttgatgagatttcacgtgcagcgtatcgccaaaacggaacctccaaggggctttcaggcatctcgggcaatggaaattctgaagatgcgctagatcgtcagacttacctgcgaaccagtgcggaaaagcagcaggcagctgttgaagagcttgatcgcctccgtacggaaaacgccaacaaggaatcggtgttgcgccaggcccgcatcgttgctgagcagcgtgaggcggaagccgtcgaaaagcaagtccagaccgaggctgcaattgccgcaaacagcgagcagctcaatgtcttgactaacaatcgcagtaccttggttgcccagcgtgatggggctgagcgcaacttggccatcgctcgtgcgcaggcggataatctgcaaggtcagcgtgctgagtacgaggaattccagcaggcagagcaggctcgcatccaggcggaagcggaagctcaggctgctgcggaggagaagcgtcgtgccgatgaggctgctgcacaggcagccgctgaagctcaagaagctgcccagcaagctcaggcggcggaggaagcccaagccgcgcaagcagctgagacagcacaagcccaagccgcgcaagctgcggaaacccaagctgcacaagccgcgcaagctcaggcagaagcgaatgatcgtgccgccgcgcaacagcgtgctgcagaggctcaagcagcagcggaacaggcgcaacgtgaggctgacgctcaggcggccaacgatgcccaagctcaggcactgcgtgaacaggcgctcaccgcagcctccatcgctgcggctgctctaattgcggcgagccagtccagccatgccactactcaaaatccttacccaactgatgaagacgcggatccgaccgatattgcggacatccaaggcccaacgcagccaggtacgggtgagtctggagattcccagagcaactccagcgacaacgattccacaggcaacgattccacaggctctgactcttcagattcagattcctccggcaacgattcttcagaggttatttccggcgatcgttccgctcagattgagactgtgattgcgcgcgccatgagccagttgggtgtgcagtacgcatggggtggcggtaacgctaatggcccaactctgggtatccgtgacggtggcgtggcggactcttacggcgattacaacaaggttggcttcgactgctctggactgaccttgtatgcgtttgcgggtgtgggaatttcacttcctcactacacgggctaccagtaccagcacggcaccaaggtgtcgccttctgagatgcaacgtggcgatctgatcttctatggtccgggagcgtctcagcacgtggcaatttacctcggtgatggtcagatgattgaggctccgaattcgggttctgtcgtgaagatttctcctgttcgctggagcggaatgaccgagagcgtggtacgcctcatttagtttcctcctatgaatcttgatgtggttcatgcgtttttatgcaatatcaaccaaaagttggtacgatcctcatatgaatgaacgcacatcggatgcatttgacgccctccttgtgctctccttcggtggtcccgaagggcacgaggaggttcgtccgtttttggagaatgtcactcacggaagggggattccgccggaacgtctagatgaagtggcggttcattaccaccacttcggtggtatcagccccatcaatgcgctgaacagggaaattatcgccaatgtggaaaaagaattggcgtctcgcgatcacaagctgcctgtttattttggtaaccgcaactggaagccgtttgataatgaggccgctgaacaaatggctgatgacggcgtgaaaaacgcgctggtgttggcaacttccgcttggggtggctactccggttgtcggcagtaccaggaagatattcagggcatgatcaagcacctggagtctcaggggcagtcgatcacgttcaccaagctgcgtcagttctacgatcaccctcgttttgtctccaccatggctcaattggttcaggattcctacgcgaagcttcccgatgagctgcgagatgaggcgcgtctggtcttcaccgcgcactccattccactgactgcggacaatgctgcgggaacccctgaggatggctccttgtattccacacaggtcaaggaagcgtcagcactgattgctgaggctgttggtgtgtcagattttgatgtggtgtggcagtcccgctcgggtagcccgcacactccgtggctggagcctgacatcgtggatcacgcagtggagctcaacgagaagggtcaaaaagcgctcgttgtctgccctgtaggctttatttctgatcatatggaagtcatttgggatcttgattccgagctgatggaagaagccgagaagcgcaacatggtggtcgagcgtgtcgctaccgttggccccaccgatgaattcgca

SEQ ID NO: 2 - protein, 630 aa, Corynebacterium glutamicum ATCC13032
MANLDSVRPGVRAALRRTHTQVKGVTLTRALIALAVSGALLSSMTPAVAQPQNPDDAAIAQAEENVSAGDGEVARLAGSLSSTDAEINRVELEMGALREEVNKSLVDLHDAQAIAEQARQDALAAKKDLDDSQAQIEAAQERLDEISRAAYRQNGTSKGLSGISGNGNSEDALDRQTYLRTSAEKQQAAVEELDRLRTENANKESVLRQARIVAEQREAEAVEKQVQTEAAIAANSEQLNVLTNNRSTLVAQRDGAERNLAIARAQADNLQGQRAEYEEFQQAEQARIQAEAEAQAAAEEKRRADEAAAQAAAEAQEAAQQAQAAEEAQAAQAAETAQAQAAQAAETQAAQAAQAQAEANDRAAAQQRAAEAQAAAEQAQREADAQAANDAQAQALREQALTAASIAAAALIAASQSSHATTQNPYPTDEDADPTDIADIQGPTQPGTGESGDSQSNSSDNDSTGNDSTGSDSSDSDSSGNDSSEVISGDRSAQIETVIARAMSQLGVQYAWGGGNANGPTLGIRDGGVADSYGDYNKVGFDCSGLTLYAFAGVGISLPHYTGYQYQHGTKVSPSEMQRGDLIFYGPGASQHVAIYLGDGQMIEAPNSGSVVKISPVRWSGMTESVVRLI

SEQ ID NO: 3 - DNA, 3815 bp, Corynebacterium glutamicum
  misc_feature (1)..(411): 3′-sequence of the cds of NCgl1481
  misc_feature (412)..(414): taa stop codon
  misc_feature (565)..(565): nucleobase thymine
  misc_feature (585)..(585): nucleobase guanine
  misc_feature (632)..(637): taatgt motif according to Pfeifer-Sancar et al.
  misc_feature (644)..(644): transcriptional start site according to Pfeifer-Sancar et al.
  CDS (923)..(2812): coding sequence
  misc_feature (1001)..(2812): coding sequence of a variant of NCgl1480
  misc_feature (1190)..(1192): atc codon for isoleucine
  misc_feature (1397)..(1399): agg codon for arginine
  misc_feature (2267)..(2269): gat codon for aspartic acid
  misc_feature (2282)..(2284): ttc codon for phenylalanine
  misc_feature (2813)..(2815): tag stop codon
  misc_feature (2889)..(3815): 5′-sequence of the cds of NCgl1479
tttccgactttggaaaaagaccttattaatgttgtgacagatgcaaaggaatcagattttggatctttaggggtcgtgattcttgatgaaactccagtgatgacttccaacctgagggatattgcgcaggagctgttgattcaaacggatctggacaccgttgtggtgcgggctccaatgtcggctgcggtggtgagtgatgttcattcgagggcggcgcttgagtcagggcagcatgatttgcttgggaccactgattacgtgctgggaacggagcttttggtacaggatgtaacggaatcgacggtggggaatattgattggggtcaattgctgatttgggggttggttgctttggcaatcgccgtggttgttgcgggtgcgtctgtgcgtcgaaaagcaatatctttataagtaaagttctaaagctttacttatagcactttcggtaggtgtcgagaaaattcccaaattgcggaaaattcacatactttgtgccacaaagtgataaacatcacaaaatttcatgactcagccattcatgcaggccagaacatgcaaaactttgcacgctctggccttttgttatgtaactgttgtgaaagatgtatcaaaagttaccaaggtgacttaatgttcagatatcgactcatgggttgttttccaaaacttgaatcaatttgagtaacagtagttatcaagcgttaaaccttgaaacttccactctttttactgatgggcatttgggcaaacggggaagcttgctggagatgcaataaatcggtgaatggttggtgcaatgaagaggagagtggcccatagtttccgctacggaacgcggagtctttacgtttcaattcttggcaagtatctagcgtcaaggcttgatatgtgaagggattcggattgaacaggagaacatgtggccaatctagattcagttcgccctggggtgcgggcagctcttcgacgtacccatacccaggtaaaaggtgtcacgttgaccagggcgttgattgcgcttgcagtaagcggagctttgcttagttccatgactccggcggtggcgcagccacagaatccggatgacgcagccattgcacaggcagaggaaaatgtttcggcgggcgatggggaagtcgcccgcctggcaggatctttgtccagcactgacgcggaaattaaccgcatcgagctggaaatgggtgctctgcgtgaagaagtgaacaagtccctcgtggatttgcatgatgcgcaggcaatcgccgagcaggcccgccaagatgcacttgcagccaagaaggatctcgatgattctcaagcgcagatcgaagcagcccaagagcgccttgatgagatttcacgtgcagcgtatcgccaaaacggaacctccaagaggctttcaggcatctcgggcaatggaaattctgaagatgcgctagatcgtcagacttacctgcgaaccagtgcggaaaagcagcaggcagctgttgaagagcttgatcgcctccgtacggaaaacgccaacaaggaatcggtgttgcgccaggcccgcatcgttgctgagcagcgtgaggcggaagccgtcgaaaagcaagtccagaccgaggctgcaattgccgcaaacagcgagcagctcaatgtcttgactaacaatcgcagtaccttggttgcccagcgtgatggggctgagcgcaacttggccatcgctcgtgcgcaggcggataatctgcaaggtcagcgtgctgagtacgaggaattccagcaggcagagcaggctcgcatccaggcggaagcggaagctcaggctgctgcggaggagaagcgtcgtgccgatgaggctgctgcacaggcagccgctgaagctcaagaagctgcccagcaagctcaggcggcggaggaagcccaagccgcgcaagcagctgagacagcacaagcccaagccgcgcaagctgcggaaacccaagctgcacaagccgcgcaagctcaggcagaagcgaatgatcgtgccgccgcgcaacagcgtgctgcagaggctcaagcagcagcggaacaggcgcaacgtgaggctgacgctcaggcggccaacgatgcccaagctcaggcactgcgtgaacaggcgctcaccgcagcctccatcgctgcggctgctctaattgcggcgagccagtccagccatgccactactcaaaatccttacccaactgatgaagacgcggatccgaccgatattgcggacatccaaggcccaacgcagccaggtacggatgagtctggagatttccagagcaactccagcgacaacgattccacaggcaacgattccacaggctctgactcttcagattcagattcctccggcaacgattcttcagaggttatttccggcgatcgttccgctcagattgagactgtgattgcgcgcgccatgagccagttgggtgtgcagtacgcatggggtggcggtaacgctaatggcccaactctgggtatccgtgacggtggcgtggcggactcttacggcgattacaacaaggttggcttcgactgctctggactgaccttgtatgcgtttgcgggtgtgggaatttcacttcctcactacacgggctaccagtaccagcacggcaccaaggtgtcgccttctgagatgcaacgtggcgatctgatcttctatggtccgggagcgtctcagcacgtggcaatttacctcggtgatggtcagatgattgaggctccgaattcgggttctgtcgtgaagatttctcctgttcgctggagcggaatgaccgagagcgtggtacgcctcatttagtttcctcctatgaatcttgatgtggttcatgcgtttttatgcaatatcaaccaaaagttggtacgatcctcatatgaatgaacgcacatcggatgcatttgacgccctccttgtgctctccttcggtggtcccgaagggcacgaggaggttcgtccgtttttggagaatgtcactcacggaagggggattccgccggaacgtctagatgaagtggcggttcattaccaccacttcggtggtatcagccccatcaatgcgctgaacagggaaattatcgccaatgtggaaaaagaattggcgtctcgcgatcacaagctgcctgtttattttggtaaccgcaactggaagccgtttgataatgaggccgctgaacaaatggctgatgacggcgtgaaaaacgcgctggtgttggcaacttccgcttggggtggctactccggttgtcggcagtaccaggaagatattcagggcatgatcaagcacctggagtctcaggggcagtcgatcacgttcaccaagctgcgtcagttctacgatcaccctcgttttgtctccaccatggctcaattggttcaggattcctacgcgaagcttcccgatgagctgcgagatgaggcgcgtctggtcttcaccgcgcactccattccactgactgcggacaatgctgcgggaacccctgaggatggctccttgtattccacacaggtcaaggaagcgtcagcactgattgctgaggctgttggtgtgtcagattttgatgtggtgtggcagtcccgctcgggtagcccgcacactccgtggctggagcctgacatcgtggatcacgcagtggagctcaacgagaagggtcaaaaagcgctcgttgtctgccctgtaggctttatttctgatcatatggaagtcatttgggatcttgattccgagctgatggaagaagccgagaagcgcaacatggtggtcgagcgtgtcgctaccgttggccccaccgatgaattcgca

SEQ ID NO: 4 - protein, 630 aa, Corynebacterium glutamicum
MANLDSVRPGVRAALRRTHTQVKGVTLTRALIALAVSGALLSSMTPAVAQPQNPDDAAIAQAEENVSAGDGEVARLAGSLSSTDAEINRIELEMGALREEVNKSLVDLHDAQAIAEQARQDALAAKKDLDDSQAQIEAAQERLDEISRAAYRQNGTSKRLSGISGNGNSEDALDRQTYLRTSAEKQQAAVEELDRLRTENANKESVLRQARIVAEQREAEAVEKQVQTEAAIAANSEQLNVLTNNRSTLVAQRDGAERNLAIARAQADNLQGQRAEYEEFQQAEQARIQAEAEAQAAAEEKRRADEAAAQAAAEAQEAAQQAQAAEEAQAAQAAETAQAQAAQAAETQAAQAAQAQAEANDRAAAQQRAAEAQAAAEQAQREADAQAANDAQAQALREQALTAASIAAAALIAASQSSHATTQNPYPTDEDADPTDIADIQGPTQPGTDESGDFQSNSSDNDSTGNDSTGSDSSDSDSSGNDSSEVISGDRSAQIETVIARAMSQLGVQYAWGGGNANGPTLGIRDGGVADSYGDYNKVGFDCSGLTLYAFAGVGISLPHYTGYQYQHGTKVSPSEMQRGDLIFYGPGASQHVAIYLGDGQMIEAPNSGSVVKISPVRWSGMTESVVRLI

SEQ ID NO: 5 - DNA, 1266 bp, Corynebacterium glutamicum ATCC13032
  CDS (1)..(1263): cds of aspartokinase gene
  misc_feature (1264)..(1266)
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaa

SEQ ID NO: 6 - protein, 421 aa, Corynebacterium glutamicum ATCC13032
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 7 - DNA, 1245 bp, Corynebacterium glutamicum ATCC13032
  CDS (1)..(1242): coding sequence of NCgl2986
  misc_feature (1243)..(1245): taa stop codon
gtggatgaactatatccattgataattttgaacatgaatgatggaaggagcagggtgtctaaagtcctgagagttggcgatcgcagcccgcgcgtggcagaagtgcgcactacgctcgctcgcctcggtgtgattgaaggctattccagggagatgtctgcaaagacagaatcccagaagttccacgaagaagagacgcttttcgacgaagaactcagcctcagcatcaagtcattccagcaagctcgaggagtcgttccctccgggcttattgacgaccccaccctgcgcgcaatccgcgaagcctcctacaccctgggaacccgcgtgctggcctaccagcccggcaaccagcttgttggtgacgacgttgtagaaatccaatcccatctccaagagctcggcttctacgccgaccgtgtggatggacattttggcgagctcacacacaaagctgtgatgaactaccaactcaactacggcatgcaggtagacggcatctgtggccctgacaccatccgtgcgctgtcccgacttggtctgcgcatcaagggtggctctgctcaagctatccgtgaacgcgaacgcatgcgcaatgcaggcccacgtcttgctggcaagcgtgtggtcattgatcctgcgcttgggggctccaacaagggtcagatcgtgaaaggcccctacggtgagatctctgaggaagaaatcctctgggatttggccacccgcctggaaggtcgcatgatcgcaacaggcatggaaaccattctgtcgcgcccgcacatggatgatcccagcagccgtgatcgcgcgtcgatcgcgaatgctttcggcgctgacctcatgctgagcctgcactgcgattcctacccgaatgaaaaagctaacggcgtggccagcttctacttcggttcggaaaacggcaccaactccttgaccggtgaaacgctctccgcgtacatccaaaaagagatcgttgcccgcaccccactgaacaactgtggcagccatgcccgtacctgggatctgctgcgcctcacgcgcatgcccatggtggaagttgtcaccggttacctcaccaaccccgatgacctggcagttctgactgatccacaaatgcgtgatcacattgccgaagccatcgttgtcgccgtcaagcgcctgtacctccttgatgaggaagcacagcccaagaccggaaccttcaagttctctgagctgttgcaatcagagcaggctggctaa

SEQ ID NO: 8 - protein, 414 aa, Corynebacterium glutamicum ATCC13032
MDELYPLIILNMNDGRSRVSKVLRVGDRSPRVAEVRTTLARLGVIEGYSREMSAKTESQKFHEEETLFDEELSLSIKSFQQARGVVPSGLIDDPTLRAIREASYTLGTRVLAYQPGNQLVGDDVVEIQSHLQELGFYADRVDGHFGELTHKAVMNYQLNYGMQVDGICGPDTIRALSRLGLRIKGGSAQAIRERERMRNAGPRLAGKRVVIDPALGGSNKGQIVKGPYGEISEEEILWDLATRLEGRMIATGMETILSRPHMDDPSSRDRASIANAFGADLMLSLHCDSYPNEKANGVASFYFGSENGTNSLTGETLSAYIQKEIVARTPLNNCGSHARTWDLLRLTRMPMVEVVTGYLTNPDDLAVLTDPQMRDHIAEAIVVAVKRLYLLDEEAQPKTGTFKFSELLQSEQAG

SEQ ID NO: 9 - protein, 180 aa, Corynebacterium glutamicum
  MISC_FEATURE (1)..(180): N terminus of the polypeptide identified by locus_tag cgR_1596
MANLDSVRPGVRAALRRTHTQVKGVALTRALIALAVSGALLSSMTPAVAQPQNPDDAAIAQAEENVSAGDGEVARLAGSLSSTDAEINRVELEMGALREEVNKSLVDLHDAQAIAEQARQDALAAKKDLDDSQAQIEAAQERLDEISRAAYRQNGTSKGLSGISGNGNSEDALDRQTYLR

SEQ ID NO: 10 - protein, 154 aa, Corynebacterium glutamicum
  MISC_FEATURE (1)..(154): N terminus of the polypeptide identified by locus_tag NCgl1480
MTRALIALAVSGALLSSMTPAVAQPQNPDDAAIAQAEENVSAGDGEVARLAGSLSSTDAEINRVELEMGALREEVNKSLVDLHDAQAIAEQARQDALAAKKDLDDSQAQIEAAQERLDEISRAAYRQNGTSKGLSGISGNGNSEDALDRQTYLR

SEQ ID NO: 11 - DNA, 2715 bp, artificial sequence
  polynucleotide NCgl1480*_Mut
  misc_feature (4)..(11): recognition site for restriction endonuclease SbfI
  misc_feature (2705)..(2712): recognition site for restriction endonuclease SbfI
gatcctgcaggcgtcgaaaagcaatatctttataagtaaagttctaaagctttacttatagcactttcggtaggtgtcgagaaaattcccaaattgcggaaaattcacatactttgtgccacaaagtgataaacatcacaaaatttcatgactcagccattcatgcaggccagaacatgcaaaaccttgcacgctctggccttttgttatgtaactgttgtgaaagatgtatcaaaagttaccaaggtgacttaatgttcagatatcgactcatgggttgttttccaaaacttgaatcaatttgagtaacagtagttatcaagcgttaaaccttgaaacttccactctttttactgatgggcatttgggcaaacggggaagcttgctggagatgcaataaatcggtgaatggttggtgcaatgaagaggagagtggcccatagtttccgctacggaacgcggagtctttacgtttcaattcttggcaagtatctagcgtcaaggcttgatatgtgaagggattcggattgaacaggagaacatgtggccaatctagattcagttcgccctggggtgcgggcagctcttcgacgtacccatacccaggtaaaaggtgtcacgttgaccagggcgttgattgcgcttgcagtaagcggagctttgcttagttccatgactccggcggtggcgcagccacagaatccggatgacgcagccattgcacaggcagaggaaaatgtttcggcgggcgatggggaagtcgcccgcctggcaggatctttgtccagcactgacgcggaaattaaccgcatcgagctggaaatgggtgctctgcgtgaagaagtgaacaagtccctcgtggatttgcatgatgcgcaggcaatcgccgagcaggcccgccaagatgcacttgcagccaagaaggatctcgatgattctcaagcgcagatcgaagcagcccaagagcgccttgatgagatttcacgtgcagcgtatcgccaaaacggaacctccaagaggctttcaggcatctcgggcaatggaaattctgaagatgcgctagatcgtcagacttacctgcgaaccagtgcggaaaagcagcaggcagctgttgaagagcttgatcgcctccgtacggaaaacgccaacaaggaatcggtgttgcgccaggcccgcatcgttgctgagcagcgtgaggcggaagccgtcgaaaagcaagtccagaccgaggctgcaattgccgcaaacagcgagcagctcaatgtcttgactaacaatcgcagtaccttggttgcccagcgtgatggggctgagcgcaacttggccatcgctcgtgcgcaggcggataatctgcaaggtcagcgtgctgagtacgaggaattccagcaggcagagcaggctcgcatccaggcggaagcggaagctcaggctgctgcggaggagaagcgtcgtgccgatgaggctgctgcacaggcagccgctgaagctcaagaagctgcccagcaagctcaggcggcggaggaagcccaagccgcgcaagcagctgagacagcacaagcccaagccgcgcaagctgcggaaacccaagctgcacaagccgcgcaagctcaggcagaagcgaatgatcgtgccgccgcgcaacagcgtgctgcagaggctcaagcagcagcggaacaggcgcaacgtgaggctgacgctcaggcggccaacgatgcccaagctcaggcactgcgtgaacaggcgctcaccgcagcctccatcgctgcggctgctctaattgcggcgagccagtccagccatgccactactcaaaatccttacccaactgatgaagacgcggatccgaccgatattgcggacatccaaggcccaacgcagccaggtacggatgagtctggagatttccagagcaactccagcgacaacgattccacaggcaacgattccacaggctctgactcttcagattcagattcctccggcaacgattcttcagaggttatttccggcgatcgttccgctcagattgagactgtgattgcgcgcgccatgagccagttgggtgtgcagtacgcatggggtggcggtaacgctaatggcccaactctgggtatccgtgacggtggcgtggcggactcttacggcgattacaacaaggttggcttcgactgctctggactgaccttgtatgcgtttgcgggtgtgggaatttcacttcctcactacacgggctaccagtaccagcacggcaccaaggtgtcgccttctgagatgcaacgtggcgatctgatcttctatggtccgggagcgtctcagcacgtggcaatttacctcggtgatggtcagatgattgaggctccgaattcgggttctgtcgtgaagatttctcctgttcgctggagcggaatgaccgagagcgtggtacgcctcatttagtttcctcctatgaatcttgatgtggttcatgcgtttttatgcaatatcaaccaaaagttggtacgatcctcatatgaatgaacgcacatcggatgcatttgacgccctccttgtgctctccttcggtggtcccgaagggcacgaggaggttcgtccgtttttggagaatgtcactcacggaagggggattccgccggaacgtctagatgaagtggcggttcattaccaccacttcggtggtatcagccccatcaatgcgctgaacaggcctgcaggatc

SEQ ID NO: 12 - DNA, 20 bp, artificial sequence
  primer LC-NCgl1480_1
agcggagctttgcttagttc

SEQ ID NO: 13 - DNA, 20 bp, artificial sequence
  primer LC-NCgl1480_2
tgcttcgatctgcgcttgag

SEQ ID NO: 14 - DNA, 20 bp, artificial sequence
  probe NCgl1480_90_C
ccagctcgatgcggttaatt

SEQ ID NO: 15 - DNA, 35 bp, artificial sequence
  probe NCgl1480_90_A
agggacttgttcacttcttcacgcagagcacccat

SEQ ID NO: 16 - DNA, 20 bp, artificial sequence
  primer LC-NCgl1480_3
cgctgcggctgctctaattg

SEQ ID NO: 17 - DNA, 20 bp, artificial sequence
  primer LC-NCgl1480_4
gcgtactgcacacccaactg

SEQ ID NO: 18 - DNA, 20 bp, artificial sequence
  probe NCgl1480_454_C
gttgctctggaaatctccag

SEQ ID NO: 19 - DNA, 35 bp, artificial sequence
  probe NCgl1480_454_A
ctgtggaatcgttgcctgtggaatcgttgtcgctg

SEQ ID NO: 20 - DNA, 1326 bp, Corynebacterium glutamicum
atccggatgacgcagccattgcacaggcagaggaaaatgtttcggcgggcgatggggaagtcgcccgcctggcaggatctttgtccagcactgacgcggaaattaaccgcatcgagctggaaatgggtgctctgcgtgaagaagtgaacaagtccctcgtggatttgcatgatgcgcaggcaatcgccgagcaggcccgccaagatgcacttgcagccaagaaggatctcgatgattctcaagcgcagatcgaagcagcccaagagcgccttgatgagatttcacgtgcagcgtatcgccaaaacggaacctccaagaggctttcaggcatctcgggcaatggaaattctgaagatgcgctagatcgtcagacttacctgcgaaccagtgcggaaaagcagcaggcagctgttgaagagcttgatcgcctccgtacggaaaacgccaacaaggaatcggtgttgcgccaggcccgcatcgttgctgagcagcgtgaggcggaagccgtcgaaaagcaagtccagaccgaggctgcaattgccgcaaacagcgagcagctcaatgtcttgactaacaatcgcagtaccttggttgcccagcgtgatggggctgagcgcaacttggccatcgctcgtgcgcaggcggataatctgcaaggtcagcgtgctgagtacgaggaattccagcaggcagagcaggctcgcatccaggcggaagcggaagctcaggctgctgcggaggagaagcgtcgtgccgatgaggctgctgcacaggcagccgctgaagctcaagaagctgcccagcaagctcaggcggcggaggaagcccaagccgcgcaagcagctgagacagcacaagcccaagccgcgcaagctgcggaaacccaagctgcacaagccgcgcaagctcaggcagaagcgaatgatcgtgccgccgcgcaacagcgtgctgcagaggctcaagcagcagcggaacaggcgcaacgtgaggctgacgctcaggcggccaacgatgcccaagctcaggcactgcgtgaacaggcgctcaccgcagcctccatcgctgcggctgctctaattgcggcgagccagtccagccatgccactactcaaaatccttacccaactgatgaagacgcggatccgaccgatattgcggacatccaaggcccaacgcagccaggtacggatgagtctggagatttccagagcaactccagcgacaacgattccacaggcaacgattccacaggctctgactcttcagattcagattcctccggcaacgattcttcagaggttatttccggcgatcgttccgctcaga